(19) United States
(12) Patent Application Publication
(10) Pub. No.: US 2015/0017108 A1
(43) Pub. Date: Jan. 15, 2015

(54) METHOD OF INHIBITING THE FORMATION OF DENTAL PLAQUE

(71) Applicant: CHUN-ERH WANG, Taipei City (TW)

(72) Inventor: CHUN-ERH WANG, Taipei City (TW)

(21) Appl. No.: 14/498,393

(22) Filed: Sep. 26, 2014

Related U.S. Application Data
(62) Division of application No. 11/710,518, filed on Feb. 26, 2007.

(30) Foreign Application Priority Data
Sep. 15, 2006 (TW) 095134362

(51) Int. Cl. A61K 8/97; A61Q 11/00; A61K 8/34

(52) U.S. Cl. CPC A61K 8/97; A61K 8/345; A61K 8/34; A61Q 11/00; A61K 2800/92; A61K 2800/30
USPC 424/58

(57) ABSTRACT

A method of inhibiting the formation of dental plaque comprises staying an oral cavity cleaning composition in oral cavity for at least 30 seconds, 5 times a day, for a total period of 2 weeks, wherein, the oral cavity cleaning composition, comprising 35˜50 parts by weight of tea leaves extract, 10˜25 parts by weight of Stevia leaf extract, 10˜25 parts by weight of lemon extract, 10˜15 parts by weight of mint leaf extract, 10˜20 parts by weight of Lonicerae Flos extract, 20˜30 parts by weight of kuding tea leaf extract, 5˜10 parts by weight of xylitol and 25˜35 parts by weight of ethanol.

All ingredients in the composition are obtained from edible plants that are very effective in oral cavity care. Furthermore, the composition is non-irritant to the body and can be used for long term.

### CROSS-REFERENCE TO RELATED APPLICATION

This application is a divisional patent application of U.S. application Ser. No. 11/710,518 filed on Feb. 26, 2007, the entire contents of which are hereby incorporated by reference for which priority is claimed under 35 U.S.C. §120.

### FIELD OF THE INVENTION

The present invention provides a method of inhibiting the formation of dental plaque, especially a method of inhibiting the formation of dental plaque using oral cleaning composition of plant origin.

### BACKGROUND OF THE INVENTION

As oral cavity hygiene cause much concern in recent years, the products related to oral cavity care become more and more diverse. The common oral cavity care products are aimed to elimination of bad breath, inhibition of formation of dental plaque or prevention of dental caries.

Bad breath is typically a consequence of volatile sulfur compounds (VSCs), which are produced by degradation of food debris depositing between teeth and periodontal sac by microorganisms. Dental plaque is a plaque formed by polysaccharide produced by bacterial metabolism in oral cavity and bacteria embedded in said polysaccharide. Dental plaque is very adhesive and uneasy to remove, thus it is usually the main cause of periodontal diseases and dental caries. In view of the above, addition of antibacterial agents to oral cavity cleaning products can be expected useful in eliminating bad breath, inhibiting formation of dental plaque and preventing dental caries.

In the numerous oral cavity products, mouthwash is helpful in oral cleaning and convenient to use, especially for patients receiving dental surgery or cervical surgery; therefore mouthwash is one of the popular consumers' products. For example, the mouthwashes of Listerine series produced by Pfizer Pharmaceutical Company comprise thymol as the active ingredient. Thymol can alter the cell walls of bacteria and thus has antibacterial effect, but it causes tooth stain and has bitter taste, in addition, it may cause some side effects, such as burning feeling in oral cavity etc.

Another common antibacterial ingredient in mouthwash products (for example, Day And Night mouthwash) is chlorhexidine (CHX). Chlorhexidine, at low concentration, exerts bacteriostatic action by adsorbing onto the cell walls of bacteria and causing leakage of cytoplasma therefrom; and at high concentration, exerts bacteriocidal action by precipitating and aggregating the cell content of bacteria. However, the products containing chlorhexidine have disadvantages such as tooth and tongue stain, bad taste, temporary taste disturbance, temporary epithelial detaching and increased deposit of dental calculus.

From the above, it can be seen that the presently available mouthwash products, although have some antibacterial effects, may cause discomfort to the patients and may damage to the mucous membrane of oral cavity if they stay in oral cavity for too long time. In addition, some chemical ingredients contained in mouthwash products may be harmful to human body if used for long term. Furthermore, if the content of ethanol in a mouthwash product reaches up to 25% or more, said product may become irritant to the mucous membrane of the oral cavity and hence are not suitable for long-term use, especially for the persons with oral cavity lesions. Therefore, a mouthwash product that does not contain any harmful or irritant ingredients and is suitable for long-term use is desired.

### SUMMARY OF INVENTION

In order to improve the existing mouthwash products, the object of the present invention is to provide an oral cavity cleaning composition that is prepared form edible natural plants and does not contain any ingredients irritating or harmful to the human body; therefore, said composition can be frequently used.

The method of inhibiting the formation of dental plaque according to the present invention, comprising: staying an oral cavity cleaning composition in oral cavity for at least 30 seconds, 5 times a day, for a total period of 2 weeks.

The oral cavity cleaning composition according to the present invention comprises 35˜50 parts by weight of tea leaves extract, 10˜25 parts by weight of Stevia leaf extract, 10˜25 parts by weight of lemon extract, 10˜15 parts by weight of mint leaf extract, 10˜20 parts by weight of Lonicerae Flos extract, 20˜30 parts by weight of kuding tea leaf extract, 5˜10 parts by weight of xylitol and 25˜35 parts by weight of ethanol.

The tea leaves used in the present invention may be dried tea leaves, or baked tea leaves produced by fermenting fresh tea leaves by a conventional fermentation method used in tea-making industry and then baking the fermented tea leaves. There are no special limitations on the kinds of the tea leaves, the tea leaves preferably comprise Pu-erh tea leaf; more preferably comprise Pu-erh tea leaf and green tea leaf or comprise Pu-erh tea leaf, black tea leaf and green tea leaf; most preferably comprise Pu-erh tea leaf, green tea leaf, black tea leaf, Ti-Kuan-Yin tea leaf, Oolong tea leaf and oiltea leaf.

In the preferred embodiment of the present invention, the tea leaves comprise 2.4˜3.6 parts by weight of Pu-erh tea leaf, 2.4˜3.6 parts by weight of green tea leaf, 0.8˜1.2 parts by weight of black tea leaf, 0.8˜1.2 parts by weight of Ti-Kuan-Yin tea leaf, 0.8˜1.2 parts by weight of Oolong tea leaf and 0.8˜1.2 parts by weight of oiltea leaf.

Said tea leaves extract is prepared by pulverizing tea leaves, mixing the pulverized tea leaves with a mixture of water and ethanol for a period of time sufficient to allow dissolution of the active ingredients, separating the obtained extract from the pulverized tea leaves, and removing ethanol and the components which may cause turbidity of the product (such as theophylline, tea polysaccharide, chlorophyll etc.) from the extract. Preferably, the extract after removing the components that may cause turbidity of the product is further concentrated to 30˜50% by weight. The removal of the components that may cause turbidity of the product is preferably achieved by adsorbing these components by a resin. Such removing method is superior to the conventional method of extraction with ethyl acetate because the latter method has lower extraction efficiency, need more raw material and may result in malodor of the product. In addition, ethyl acetate, which is an organic solvent, is harmful to human health if large amount is ingested.

In order to effectively extract the active ingredients from the tea leaves, the extract can be mixed with the same pulverized tea leaves used before to perform extraction again. The extraction step can be repeated as such for one or several times.

In the process of preparation of the tea leaves extract according to the present invention, the weight ratio of tea leaves:water:ethanol in the step of extraction with a mixture of water and ethanol is 0.8˜1.2: 0.6˜0.9: 0.2˜0.3.

The Stevia leaf extract according to the present invention is prepared by drying and pulverized Stevia leaves, mixed the pulverized Stevia with a mixture of water and ethanol for a period of time sufficient to allow dissolution of the active ingredients, separating the obtained extract from the pulverized Stevia and removing ethanol from the extract. The lemon extract, the mint leaf extract, the Lonicerae Flos extract and the kuding tea leaf extract according to the present invention are prepared in the same manner as stated above.

The weight ratio of any one of the aforesaid plants other than tea leaves (i.e. Stevia leaf, lemon, mint leaf, Lonicerae Flos and kuding tea leaf): water: ethanol is 0.8˜1.2: 2.4˜3.6: 1.6˜2.4.

In the preferred embodiment of the present invention, all the aforesaid extracts are those wherein ethanol has been removed.

The effects of the ingredients contained in the oral cavity cleaning composition according to the present invention are described below.

Tea leaves extract is the main ingredient of the composition according to the present invention. The tea leaves contain fluorine, which can enhance the acid resistance of enamel of teeth because fluorine can replace hydroxyl groups of hydroxyapatite to form fluorapatite. In addition, catechins contained in tea have antibacterial and anti-inflammatory effects and have been proved effective in reducing dental plaque and periodontal disease index in clinics; thus can provide beneficial effects in oral cavity care.

Stevia leaf contains stevioside. Stevioside is not liable to degradation and utilization by microorganisms and hence has less tendency to cause dental plaque and growth of bacteria which are responsible for dental caries. Furthermore, stevioside produces less calories, in addition, has sweetness as 300 times as sugar such that the composition of the present invention has good palatability.

Lemon has high content of vitamin C. Vitamin C is an important nutrient for maintaining gingival health. Severe deficiency of vitamin C may result in weakness, swelling, bleeding and vulnerability of gum, and loosening or loss of teeth. Intake of suitable amount of vitamin C may produce whitening effect. Besides, addition of lemon extract can improve the flavor of the composition of the present invention.

The flavor of mint leaf extract is helpful in reviving spirit and refreshing breath. The monoterpine compounds contained in the mint leaf extract can reach lung through blood circulation and hence can improve the odor of breath. The mouthwash containing mint leaf extract can relieve gingival inflammation and swelling, and reduce bacterial growth in oral cavity.

Xylitol is very helpful in prevention of dental caries. The microorganisms in oral cavity can utilize the ingested hydrocarbons or sugar and produce acidic substances, resulting in reduction of the pH of oral cavity to 5.7. Enamel of teeth becomes damaged in such acidic environment and leads to formation of dental caries. Xylitol has acid-neutralizing ability and hence can maintain acid-base balance in oral cavity and prevent dental caries. Xylitol used in the present invention may be commercially available or synthesized by the conventional methods known in the art.

Lonicerae Flos has heat-removing, detoxifying, anti-inflammatory, blood-clearing and bacteriocidal effects and has been used against viruses and bacteria since ancient times. For example, among the royal formulations of Ching dynasty, one formulation containing Lonicerae Flos and other herbs was used as mouthwash for periodontal diseases and canker.

Kuding tea has heat-removing, anti-inflammatory and analgesic effects, and is effective in treatment of headache, cough, toothache and swelling throat caused by heat evils as well as helpful in oral cavity and throat care.

From the aforesaid effects of the ingredients contained in the composition according to the present invention, it can be known that these ingredients are all from edible plants and helpful in maintaining the health of oral cavity, gum and throat. Furthermore, the composition according to the present invention has lower ethanol content than the commercially available products and has good flavor and palatable taste without irritation to the mucous membrane of oral cavity; therefore, even the persons with oral cavity lesions, periodontis, sensitive and sore tooth, or gum bleeding, can use it.

The oral cavity cleaning composition according to the present invention is further described by the following Examples. The Examples is merely intended to illustrate the present invention but not to limit its scope. Theses Examples can be altered or modified by the persons having ordinary skills in the art without departing the spirit and the scope of the present invention.

### DETAILED DESCRIPTION OF THE PREFERRED EMBODIMENTS

### Example 1 Preparation of Oral Cavity Cleaning Composition According to the Present Invention

Preparation Of Tea Leaves Extract

The fresh Pu-Erh tea leaf, green tea leaf, black tea leaf, Ti-Kuan-Yin tea leaf, Oolong tea leaf and oiltea leaf were fermented respectively. The fermented tea leaves were mixed in a weight proportion of Pu-Erh tea leaf 3 parts, green tea leaf 3 parts, black tea leaf 1 part, Ti-Kuan-Yin tea leaf 1 part, Oolong tea leaf 1 part and oiltea leaf 1 part (total weight: 2000 kg), and then baked and pulverized. To the pulverized tea leaves, a mixture of 500kg of ethanol and 2500 kg of water were added and the resulting mixture was sealed for 4 hours. The liquid was separated from the pulverized tea leaves by filtration and then distilled at the temperature of 65° C. to remove ethanol. The liquid after removing ethanol was treated with an adsorbing resin to remove the components that may cause turbidity of the product, for example, theophylline, tea polysaccharides and chlorophyll etc., and then recovered. To the recovered liquid, 500 kg of ethanol and the pulverized tea leaves that have subjected to the first extraction were added and mixed for 8 hours to perform the second extraction, and the subsequent treatment were carried out in the same manner as stated in the first extraction. To the liquid recovered form the second extraction, ethanol and the pulverized tea leaves that have subjected to the first and second extraction were added and mixed for 24 hours to perform the third extraction, and the subsequent treatment were carried out in the same manner as stated in the first extraction. The liquid recovered form the third extraction was concentrated at low temperature to 40 wt % based on said liquid recovered form the third extraction. The tea leaves extract thus obtained was stored until use.

Preparation Of Other Plant Extracts

100 kg of Stevia leaves were baked dry and pulverized. To the pulverized Stevia leaves, 200 kg of ethanol and 300 kg of water were added and mixed for 6 hours and then filtered. The liquid was separated from the pulverized Stevia leaves and distilled at the temperature of 65° C. to remove ethanol. Thereby, the Stevia leaf extract was obtained.

Other plant extracts, such as lemon extract, mint leaf extract, Lonicerae Flos extract and kuding tea leaf extract were obtained in the same manner as stated in preparation of the Stevia leaf extract.

Preparation Of The Composition According To The Present Invention

The composition according to the present invention was prepared by mixting 35˜50 parts by weight of tea leaves extract, 10˜25 parts by weight of Stevia leaf extract, 10˜25 parts by weight of lemon extract, 10˜15 parts by weight of mint leaf extract, 10˜20 parts by weight of Lonicerae Flos extract, 20˜30 parts by weight of kuding tea leaf extract, 5˜10 parts by weight of xylitol and 25˜35 parts by weight of ethanol.

### Example 2

Dental plaque is one of the main causes leading to dental caries and periodontal diseases (including gum diseases). Streptococcus mutans is one of the main bacteria that are responsible for the formation of dental plaque in the early stage. Streptococcus mutans can produce glucosyltransferase (GTF), which can convert sucrose to water-insoluble, extracellular polysaccharide. Said polysaccharide will facilitate bacterial adherence to the surface of teeth. Therefore, prevention of bacterial adherence to the surface of teeth is an effective approach of inhibiting the formation of dental plaque, which in turn, will prevent the occurrence of dental caries, gingivitis and periodontal diseases. In view of the above, the amount of Streptococcus mutans in the samples was used as an indicator for the effects of the composition according to the present invention in this Example.

The following test was entrusted to Super Laboratory Company to perform; wherein the oral cavity cleaning composition obtained from Example 1 was used.

Subjects Receiving The Test

Two men aged 25˜31 and eight women aged 24 to 30 (total, 10 persons; average age, 27.2±2.4) were tested. All subjects receiving the test were non-smoking, healthy adults with 10⁵/ml or more of streptococcus mutans in saliva.

Design Of The Test

The test was conducted for 4 weeks and examination was performed for 3 times during the period of the test. The first examination was intended to select the suitable subjects for receiving the test; the second examination was intended to determine the baseline of streptococcus mutans in the samples taken from the dental plaque or saliva of tested subjects; and the third examination was intended to determine the amount of streptococcus mutans in the samples taken from the dental plaque or saliva of the tested subjects who have used the oral cavity cleaning composition from Example 1 for 2 weeks. The interval between the first and the second examinations was 2 weeks and no oral cavity cleaning composition was used during this period. The interval between the second and the third examinations was also 2 weeks and the oral cavity cleaning composition was used every day during this period.

In the period of the test, the tested subjects maintained the usual diet and the oral cavity cleaning habit. However, no mouthwash was allowed to use and the toothpaste, if used, should remain the same brand as before. In addition, the tested subjects were not allowed to brush teeth or use any dental floss from 48 hours before the second or the third examination until the examination was completed. The oral cavity habit was restored after completion of the examination.

The selected subjects for receiving the test should be subjected to teeth cleaning in a dental clinic and the next day of teeth cleaning was designated as the first day of the test.

The second examination was performed 2 weeks after the test began. The samples were taken from the dental plaque or the saliva of the tested subjects and each was subjected to a series of dilution with 0.05 M phosphate buffer solution. The diluted samples were smeared on MSB (Mitis salivarius bacitracin) agar medium (Difico) and BHI (brain heart infusion) agar medium(Difico).

The tested subjects began to use the oral cavity cleaning composition from the beginning of the third week of the test. The composition was used in an amount of 20 ml each time (staying in oral cavity for 30 seconds), 5 times a day (after meals and before bedtime), for a total period of 2 weeks. At the end of the fourth week, the third examination was performed.

Examined Items

1. The Amount of Streptococcus Mutans

The tested subjects were forbidden to brush teeth and use dental floss from 48 hours before the examination until the examination was completed. The sample was taken from dental plaque or the saliva by a sterile disposable dental tool, then weighed and subjected to a series of dilution with 0.05 M phosphate buffer solution. The sample with suitable dilution ratio was inoculated on the MSB agar medium and incubated at 37° C. under an anaerobic condition for 3 days.

2. The Total Amount of All Bacteria

The total amount of all bacteria in the sample was determined by the same method as for determination of the amount of Streptococcus mutans, except after a serial dilution, the sample with suitable dilution ratio was inoculated on BHI agar medium and incubated at 35±2° C. under an anaerobic condition for 5 days. The ratio of Streptococcus mutans to all bacteria in the sample taken from the dental plaque or the saliva was then calculated from the amount of Streptococcus mutans and the total amount of all bacteria.

3. Standards for Evaluation of the Effectiveness

Compare the amount of Streptococcus mutans obtained from the second examination and that from the third examination. If 50% or more of the tested subjects showed reduction in the amount of Streptococcus mutans (p<0.05), the tested oral cavity cleaning composition is considered to be effective in reducing the amount of Streptococcus mutans in the dental plaque or the saliva.

4. Test for the Antibacterial Effect of Oral Cavity Cleaning Composition

Streptococcus mutans was suspended in 0.05 M phosphate buffer solution to the concentration of 1.5×10⁸ (CFU/ml). 0.1 ml/tube of the resulting suspension was added respectively to the tube containing 10 ml of the oral cavity cleaning composition of Example 1 (test group) and the tube containing 10 ml of sterile physiological saline (control group), then mixed thoroughly for 30 seconds to allow the composition to exert its effects. The mixture was subjected to a serial dilution with 0.05 M phosphate buffer solution. 0.2 ml of diluted samples were inoculated on MSB agar mediums in duplicate and incubated at 37° C. under an anaerobic condition for 3 days. The growth of Streptococcus mutans was observed and its inhibition percentage was calculated as follows:

inhibition (%)=(residual amount of Streptococcus mutans of the control group−residual amount of Streptococcus mutans of the test group)/residual amount of Streptococcus mutans of the control group×100%

As shown in Table 1, for the samples taken from the dental plaques of the tested subjects, the average amount of Streptococcus mutans was 4.53×10⁵ CFU/mg, and its ratio relative to the total amount of all bacteria was 3.46%. After using the composition of the present invention for 2 weeks, the average amount of Streptococcus mutans was reduced to 2.51×10⁵ CFU/mg and its ratio relative to the total amount of all bacteria was reduced to 1.73%. Furthermore, after using the composition of the present invention, 60% of the tested subjects showed that both the average amount of Streptococcus mutans and its ratio relative to total amount of all bacteria were reduced.

For the samples taken from the saliva of the tested subjects, the average amount of Streptococcus mutans was 7.30×10⁶ CFU/mg and its ratio relative to the total amount of all bacteria was 1.94%. After using the composition of the present invention for 2 weeks, the average amount of Streptococcus mutans was reduced to 4.00×10⁶ CFU/mg and its ratio relative to the total amount of all bacteria was reduced to 1.00% (Please see

Table 2). Furthermore, after using the composition of the present invention, 80% of the tested subjects showed that both the average amount of Streptococcus mutans and its ratio relative to the total amount of all bacteria were reduced.

Moreover, as shown in Table 3, after Streptococcus mutans contacted with the composition of the present invention for 30 seconds, the amount of Streptococcus mutans was reduced from 5.4×10⁶ CFU/ml to 6.6×10⁵ CFU/ml in the test group, and to 2.0×10⁶ CFU/ml in the control group. The bacterial inhibition percentage was 67%. From the above results, it can be seen that the composition of the present invention can reduce the amount of Streptococcus mutans and its ratio relative to all bacteria in oral cavity. Therefore, the composition of the present invention is effective in prevention of dental caries, gingivitis and periodontal diseases. Furthermore, the ingredients contained in the composition of the present invention are obtained from natural plants and are not irritant to the human body. The composition of the present invention also has lower ethanol content compared with the commercially available products as mentioned in the above section “BACKGROUND OF THE INVENTION ” and hence can be used for long term.

TABLE 1
Change in the amount of Streptococcus mutans (S. mutans) in
the samples taken from the dental plaques before and after use of
the oral cavity cleaning composition of the present invention

| | S. mutans | S. mutans |
|---|---|---|
| | (10⁵ CFU/mg) | (%) |
| before use | 4.53 ± 1.56 | 3.46 ± 0.95^{a} |
| after use | 2.51 ± 0.56 | 1.73 ± 0.51^{a} |
| average difference | — | −34.03 ± 17.37^{b} |
| the number of the tested | 5/10^{c} | 6/10^{d} |
| subjects showing reduction | | |
| of S. mutans | | |

Mean ± S.E.M.
^{a}CFU/mg of S. mutans/CFU/mg of all bacteria
^{b}average difference = the sum of individual differences/10, wherein individual difference = [(CFU/mg of S. mutans after use/CFU/mg of S. mutans before use) − 1] × 100%
^{c}the number of the tested subjects showing reduced CFU/mg of S. mutans/the number of the total tested subjects.
^{d}the number of the tested subjects showing reduced ratio of Streptococcus mutans to all bacteria/the number of the total tested subjects.

TABLE 2
Change in the amount of Streptococcus mutans (S. mutans)
in the samples taken from the saliva before and after use of the
oral cavity cleaning composition of the present invention

| | S. mutans | S. mutans |
|---|---|---|
| | (10⁶ CFU/mg) | (%) |
| before use | 7.30 ± 3.55 | 1.94 ± 0.65^{a} |
| after use | 4.00 ± 1.18 | 1.00 ± 0.42^{a} |
| average difference | — | −20.20 ± 28.60^{b} |
| the number of the tested | 6/10^{c} | 8/10^{d} |
| subjects showing reduction | | |
| of S. mutans | | |

Mean ± S.E.M.
^{a}CFU/mg of S. mutans/CFU/mg of all bacteria
^{b}average difference = the sum of individual differences/10, wherein individual difference = [(CFU/mg of S. mutans after use/CFU/mg of S. mutans before use) − 1] × 100%
^{c}the number of the tested subjects showing reduced CFU/mg of S. mutans/the number of the total tested subjects.
^{d}the number of the tested subjects showing reduced ratio of Streptococcus mutans to all bacteria/the number of the total tested subjects.

TABLE 3
The antibacterial effect of the tested composition after
acting on Streptococcus mutans for 30 seconds

| | Initial amount | Residual amount | |
|---|---|---|---|
| | (CFU/ml) | (CFU/ml) | Inhibition % |
| Test group | 5.4 × 10⁶ | 6.6 × 10⁵ | 67 |
| Control group | 5.4 × 10⁶ | 2.0 × 10⁶ | — |

Inhibition % = (residual amount of the control group − residual amount of the test group)/residual amount of the control group × 100%

What is claimed is:

1. A method of inhibiting the formation of dental plaque, comprising:
staying an oral cavity cleaning composition in oral cavity for at least 30 seconds, 5 times a day, for a total period of 2 weeks,
wherein, the oral cavity cleaning composition, comprising 35-50 parts by weight of tea leaves extract wherein said tea leaves are selected from the group consisting of Pu-erh tea leaf, green tea leaf, black tea leaf, Ti-Kuan-Yin tea leaf, Oolong tea leaf, oil tea leaf, and mixtures thereof, 10-25 parts by weight of Stevia leaf extract, 10-25 parts by weight of lemon extract, 10-15 parts by weight of mint leaf extract, 10-20 parts by weight of Lonicerae Flos extract, 20-30 parts by weight of kuding tea leaf extract, 5-10 parts by weight of xylitol and 25-35 parts by weight of ethanol.

2. The method according to claim 1, wherein said tea leaves comprises 2.4-3.6 parts by weight of Pu-erh tea leaf, 2.4-3.6 parts by weight of green tea leaf, 0.8-1.2 parts by weight of black tea leaf, 0.8-1.2 parts by weight of Ti-Kuan-Yin tea leaf, 0.8-1.2 parts by weight of Oolong tea leaf and 0.8-1.2 parts by weight of oil tea leaf.

3. The method according to claim 1, wherein said tea leaves extract is obtained by extracting the tea leaves with a mixture of water and ethanol.

4. The method according to claim 3, wherein the weight ratio of tea leaves:water:ethanol is 0.8-1.2:0.6-0.9:0.2-0.3.

5. The method according to claim 4, wherein said tea leaves extract is obtained by extracting the tea leaves, followed by purifying the resulting extract with a resin to remove the components that may cause turbidity of the product.

6. The method according to claim 5, wherein said components that may cause turbidity of the product are theophylline, tea polysaccharide and chlorophyll.

7. The method according to claim 5, wherein said tea leaves extract, after removal of the components which may cause turbidity of the product, is further concentrated to 30-50% solids.

8. The method according to claim 1, wherein said Stevia leaf extract, said lemon extract, said mint leaf extract, said Lonicerae Flos extract and said kuding tea leaf extract are all obtained by extracting the respective plants with a mixture of water and ethanol.

9. The method according to claim 8, wherein the weight ratio of said plants:water:ethanol is 0.8-1.2:2.4-3.6:1.6-2.4.

10. The method according to claim 1, wherein said tea leaves extract, said Stevia leaf extract, said lemon extract, said mint leaf extract, said Lonicerae Flos extract and said kuding tea leaf extract are all the extracts wherein ethanol has been removed.

11. A method of inhibiting the formation of dental plaque, comprising:
staying an oral cavity cleaning composition in oral cavity for at least 30 seconds, 5 times a day, for a total period of 2 weeks,
wherein, the oral cavity cleaning composition, comprising 35-50 parts by weight of tea leaves extract wherein said tea leaves are selected from the group consisting of 2.4-3.6 parts by weight Pu-erh tea leaf, 2.4-3.6 parts by weight of green tea leaf, 0.8-1.2 parts by weight of black tea leaf, 0.8-1.2 parts by weight of Ti-Kuan-Yin tea leaf, 0.8-1.2 parts by weight of Oolong tea leaf, 0.8-1.2 parts by weight of oil tea leaf, and mixtures thereof, 10-25 parts by weight of Stevia leaf extract, 10-25 parts by weight of lemon extract, 10-15 parts by weight of mint leaf extract, 10-20 parts by weight of Lonicerae Flos extract, 20-30 parts by weight of kuding tea leaf extract, 5-10 parts by weight of xylitol and 25-35 parts by weight of ethanol
wherein said tea leaves extract is obtained by extracting the tea leaves with a mixture of water and ethanol;
wherein said Stevia leaf extract, said lemon extract, said mint leaf extract, said Lonicerae Flos extract and said kuding tea leaf extract are all obtained by extracting the respective plants with a mixture of water and ethanol; and
wherein said tea leaves extract, said Stevia leaf extract, said lemon extract, said mint leaf extract, said Lonicerae Flos extract and said kuding tea leaf extract are all the extracts wherein ethanol has been removed.

12. The method according to claim 11, wherein the weight ratio of tea leaves:water:ethanol is 0.8-1.2:0.6-0.9:0.2-0.3.

13. The method according to claim 12, wherein said tea leaves extract is obtained by extracting the tea leaves, followed by purifying the resulting extract with a resin to remove the components that may cause turbidity of the product.

14. The method according to claim 13, wherein said components that may cause turbidity of the product are theophylline, tea polysaccharide and chlorophyll.

15. The method according to claim 13, wherein said tea leaves extract, after removal of the components which may cause turbidity of the product, is further concentrated to 30-50% solids.

16. The method according to claim 11, wherein the weight ratio of said plants:water:ethanol is 0.8-1.2:2.4-3.6:1.6-2.4.
